(19) United States
(12) Patent Application Publication
(10) Pub. No.: US 2026/0369353 A1
(43) Pub. Date: Nov. 19, 2026

(54) Predicting Indel Frequencies

(71) Applicant: Sanofi, PARIS (FR)

(72) Inventors: Sourav Roy Choudhury, Cambridge, MA (US); Eugenia Lyashenko, Cambridge, MA (US); Youngji Na, Cambridge, MA (US); Tess Torregrosa, Cambridge, MA (US)

(21) Appl. No.: 19/136,690

(22) PCT Filed: Dec. 5, 2023
(86) PCT No.: PCT/US2023/082543

§ 371 Date: Jun. 6, 2025

Related U.S. Application Data
(60) Provisional application No. 63/430,827, filed on Dec. 7, 2022.

(30) Foreign Application Priority Data
May 2, 2023 (EP) 23315141.4

(51) Int. Cl. G16B 20/50; G16B 30/10

(52) U.S. Cl. CPC G16B 20/50; G16B 30/10

(57) ABSTRACT

The present disclosure relates to methods for estimating the frequency of insertions and/or deletions mediated by RNA-guided nucleases. Provided herein are methods and systems for a computational pipeline to process sequencing reads of edited polynucleotide target sequences.

### CROSS-REFERENCE TO RELATED APPLICATIONS

This application claims the benefit of U.S. Provisional Patent Application No. 63/430,827, filed on Dec. 7, 2022 and EP Patent Application No. EP23315141.4, filed May 2, 2023, the contents of which are incorporated herein by reference in their entirety.

### TECHNICAL FIELD

This disclosure relates to quantification of insertions and/or deletions in the vicinity of cleavage sites within a polynucleotide target sequence.

### BACKGROUND

Nucleic acid-guided nucleases can be used to edit polynucleotide sequences, for example a genome of an organism, at targeted locations with high precision. Nucleases are enzymes capable of cleaving the phosphodiester bonds between nucleotides of nucleic acids. Genome editing methods include use of CRISPR (clustered regularly interspaced short palindromic repeats)-associated proteins or similar nucleic acid-guided nucleases to induce DNA double-strand breaks (DSBs) at predictable genomic positions relative to the user-designated target sequence. DNA DSBs are repaired by intracellular machinery, for example, by non-homologous end joining (NHEJ). The repair process can result in sequence variants including, for example, insertions and deletions (indels). Quantification of the frequency of indels at the target cleavage site within an edited cell population is important for evaluating the efficacy of a nucleic-acid guided nuclease editing system.

A wide variety of nucleic-acid guided nucleases are now well known, with additional naturally-occurring and engineered nucleases being discovered and characterized. Novel nucleases may be poorly characterized, i.e. their cleavage site and editing window relative to the user-designated target sequence being unknown. Existing indel quantification data analysis pipelines for Next Generation Sequencing (NGS) rely on the assumption that cleavage site and editing window are established, which is not the case for novel poorly characterized nucleases. As a result, existing computations tools can undercount indels at the target cleavage site within an edited cell population. Few computational methods have been developed so far to address this need.

### SUMMARY

The present disclosure is based, in part, on the discovery that a data analysis pipeline can be configured to quantify insertion and deletions of a target polynucleotide sequence that has been cleaved by a nucleic-acid guided nuclease, even if the cleavage site of the nuclease relative to the user-designated target sequence is not known. The methods disclosed herein include a computational pipeline for indel quantification for polynucleotide sequences that have been cleaved by nucleic-acid guided nucleases, including uncharacterized nucleases. The methods disclosed herein include parameters for alignment of next-generation sequencing (NGS) reads from targeted amplicon sequencing (TAS) of nuclease-edited or control samples that have been obtained and aligned to reference amplicon sequences. Disclosed herein are exemplary experimental and computational data demonstrating that the methods can be used to accurately quantify indels of novel uncharacterized nucleases, for example, nucleases having unknown cleavage sites relative to the user-designated target sequence.

In some embodiments, provided herein are methods for quantifying insertions and/or deletions in a polynucleotide sequence caused by cleavage of a target polynucleotide sequence by a nucleic acid-guided nuclease. In some embodiments, the methods include, in a computer system, receiving sample sequence data comprising a plurality of sequencing reads; filtering the plurality of sequencing reads; aligning the plurality of sequencing reads to a reference sequence; defining a window based on a sequence location within a nucleic acid guide sequence and the locations of the ends of the nucleic acid guide sequence; determining, based on the alignment of each sequencing read of the plurality of sequencing reads to the reference sequence, the number of sequencing reads comprising an insertion or deletion within the window relative to the reference sequence; estimating, based on the number of sequencing reads comprising an insertion or deletion, the quantity of insertions and/or deletions in the polynucleotide sequence mediated by a nucleotide-directed nuclease. In some embodiments, the nucleic acid-guided nuclease is a Class 2 nuclease. In some embodiments, the nucleic acid-guided nuclease is a type II nuclease. In some embodiments, the nucleic acid-guided nuclease is SpCas9. In some embodiments, the nucleic acid-guided nuclease is AsCas12a. In some embodiments, the nucleic acid-guided nuclease is a type V nuclease. In some embodiments, the nucleic acid guide is a guide RNA (gRNA). In some embodiments, the nucleic acid guide is a single guide RNA (sgRNA).

In some embodiments, the center of the window is located at the center of the nucleic acid guide sequence. In some embodiments, the center of the window is located at the site where the nuclease cleaved the polynucleotide sequence. In some embodiments, the length of the window is equivalent to the length of the nucleic acid guide sequence. In some embodiments, the 5′ end of the window extends 50 basepairs 5′ to the 5′ end of the nucleic acid guide and the 3′ end of the window extends 50 basepairs 3′ to the 3′ end of the nucleic acid guide. In some embodiments, the 5′ end of the window extends 40 basepairs 5′ to the 5′ end of the nucleic acid guide and the 3′ end of the window extends 40 basepairs 3′ to the 3′ end of the nucleic acid guide. In some embodiments, the 5′ end of the window extends 30 basepairs 5′ to the 5′ end of the nucleic acid guide and the 3′ end of the window extends 30 basepairs 3′ to the 3′ end of the nucleic acid guide. In some embodiments, the 5′ end of the window extends 20 basepairs 5′ to the 5′ end of the nucleic acid guide and the 3′ end of the window extends 20 basepairs 3′ to the 3′ end of the nucleic acid guide. In some embodiments, the 5′ end of the window extends 10 basepairs 5′ to the 5′ end of the nucleic acid guide and the 3′ end of the window extends 10 basepairs 3′ to the 3′ end of the nucleic acid guide. In some embodiments, the 5′ end of the window extends 5 basepairs 5′ to the 5′ end of the nucleic acid guide and the 3′ end of the window extends 5 basepairs 3′ to the 3′ end of the nucleic acid guide.

In some embodiments, the method further includes trimming of adapter sequences from the plurality of sequencing reads. In some embodiments, the plurality of sequencing reads are generated from targeted amplicon sequencing. In some embodiments, the plurality of sequencing reads are generated from targeted amplicon sequencing of DNA isolated from cells edited by the nucleic-acid guided nuclease. In some embodiments, the plurality of sequencing reads are paired-end sequencing reads. In some embodiments, the method further includes read merging of the paired-end sequencing reads to produce a single read for alignment to the reference sequence. In some embodiments, the read merging of the paired-end reads further includes applying a minimum paired-end read overlap score to the plurality of sequencing reads. In some embodiments, the minimum paired-end read overlap score is 0, 10, 20, 30, 40, 50, 60, 70, 80, 90, or 100. In some embodiments, the minimum paired-end read overlap score is 10. In some embodiments, the read merging of paired-end reads further comprises applying a maximum paired-end read overlap score to the plurality of sequencing reads. In some embodiments, the maximum paired-end read overlap score is 100, 110, 120, 130, 140, 150, 160, 170, 180, 190, 200, 210, 220, 230, 240, 250, 260, 270, 280, 290, or 300. In some embodiments, the maximum paired-end read overlap score is 100. In some embodiments, the filtering step further comprises applying a minimum average read quality score to the plurality of sequencing reads. In some embodiments, the minimum average read quality score is 0, 5, 10, 15, 20, 25, 30, 35, or 40. In some embodiments, the filtering step further comprises applying a minimum single basepair score to the plurality of sequencing reads. In some embodiments, the minimum single basepair score is 0, 1, 2, 3, 4, 5, 6, 7, 8, 9, 10, 11, 12, 13, 14, 15, 16, 17, 18, 19, or 20. In some embodiments, the aligning step further comprises applying an amplicon minimum alignment score to the plurality of sequencing reads. In some embodiments, the amplicon minimum alignment score is 0, 10, 20, 30, 40, 50, 60, 70, 80, 90, or 100.

In some embodiments, the nucleic acid-guided nuclease is a FokI nuclease. In some embodiments, the FokI nuclease is fused to a transcription activator-like (TAL) protein. In some embodiments, the nucleic acid-guided nuclease is a zinc-finger nuclease.

In some embodiments, described herein is a computer program product tangibly embodied on a computer-readable medium, comprising instructions that when executed by one or more processors are configured to: receive sample sequence data comprising a plurality of sequencing reads; filter the plurality of sequencing reads; align the plurality of sequencing reads to a reference sequence; define a window based on a sequence location within a nucleic acid guide sequence and the locations of the ends of the nucleic acid guide sequence; determine, based on the alignment of each sequencing read of the plurality of sequencing reads to the reference sequence, the number of sequencing reads comprising an insertion or deletion within the window relative to the reference sequence; and estimate, based on the number of sequencing reads comprising an insertion or deletion, the quantity of insertions and/or deletions in the polynucleotide sequence mediated by a nucleotide-directed nuclease.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a cartoon schematic of a Type II nuclease, featuring an sgRNA interacting with a target DNA sequence. PAM (NGG) sequence, guide sequence, target DNA sequence, and cleavage site are indicated.

FIG. 1B is a cartoon schematic of a Type V nuclease, featuring a gRNA interacting with a target DNA sequence. PAM (TTTN) sequence, guide sequence, target DNA sequence, and cleavage site are indicated.

FIG. 2A is a diagram of an upstream molecular biology workflow for the methods disclosed herein. Steps include: design primers to amplify editing target; isolate cellular DNA; PCR-amplify editing target with adapters; add next-generation sequencing (NGS) adapters to PCR products; pool barcoded amplicons; bead-purify amplicons; spike in diversity DNA; perform NGS on sequencing instrument.

FIG. 2B is a diagram of the computational pipeline disclosed herein. Steps include quality control of received sequencing reads; alignment of sequencing reads to a target sequence; and quantification of indels according to the methods disclosed herein.

FIG. 3A is a schematic representation of received sequencing reads of nucleic-acid guided nuclease edited sample, comprising a plurality of sequencing reads, a subset of which contain indels as a result of cleavage by the nucleic-acid guided nuclease. The nuclease is Cas9, a Type II nuclease with a known cleavage site relative to the guide sequence. For each type of indel, the number of sequencing reads comprising that indel is indicated at right. The previously known cleavage site is indicated by a rectangular box.

FIG. 3B is a schematic representation of received sequencing reads of nucleic-acid guided nuclease edited sample, comprising a plurality of sequencing reads, a subset of which contain indels as a result of cleavage by the nucleic-acid guided nuclease. The nuclease is a novel Type V nuclease. For each type of indel, the number of sequencing reads comprising that indel is indicated at right. Several indels are missed by standard computational processing methods.

FIG. 3C is a schematic representation of received sequencing reads of nucleic-acid guided nuclease edited sample, comprising a plurality of sequencing reads, a subset of which contain indels as a result of cleavage by the nucleic-acid guided nuclease. The nuclease is a novel Type V nuclease. For each type of indel, the number of sequencing reads comprising that indel is indicated at right. Custom data processing methods capture all indels generated by cleavage by the nuclease.

FIG. 4A is a plot comparing a standard data processing pipeline for a known nuclease, Cas9, with the data processing pipeline disclosed herein for Cas9.

FIG. 4B is schematic of a dilution experiment to validate the data processing pipeline disclosed herein, wherein nucleic acid-guided nuclease-edited target sequence is serially diluted with non-edited target sequence in order to evaluate the efficacy of the data processing methods disclosed herein.

FIG. 4C is a plot of expected indel percentage (x-axis) vs. observed indel percentage (y-axis) for the experiment depicted in the schematic of FIG. 4B.

FIG. 5 is a diagram of computer system components that can be used to implement a computational pipeline for indel quantification for polynucleotide sequences that have been cleaved by nucleic-acid guided nucleases, including uncharacterized nucleases.

FIG. 6 is a plot of indel percentage (y-axis) for two nucleases (x-axis): Cas9 (reference) and novel Type V nuclease with escalating doses of the novel nuclease using two different guide sequences, A1 and F2.

FIG. 7A is a schematic of an experiment where frequency of a 21-nucleotide insertion was quantified by the computational pipeline disclosed herein.

FIG. 7B is a schematic representation of received sequencing reads of the amplicons depicted in FIG. 7A, comprising a plurality of sequencing reads, a subset of which contain the 21-nucleotide insertion in a spike-in dilution experiment.

FIG. 7C is a schematic representation of received sequencing reads of the amplicons depicted in FIG. 7A, comprising a plurality of sequencing reads, a subset of which contain the 21-nucleotide insertion in a spike-in dilution experiment.

FIG. 7D is a schematic representation of received sequencing reads of the amplicons depicted in FIG. 7A, comprising a plurality of sequencing reads, a subset of which contain the 21-nucleotide insertion in a spike-in dilution experiment.

### DETAILED DESCRIPTION

### Definitions

As used herein, the terms “nucleic acid,” “polynucleotide,” and “oligonucleotide” are interchangeable and refer to a deoxyribonucleotide or ribonucleotide polymer, in linear or circular conformation, and in either single- or double-stranded form. For the purposes of the present disclosure, these terms are not to be construed as limiting with respect to the length of the polymer. The terms can encompass known analogues of natural nucleotides, as well as nucleotides that are modified in the base, sugar and/or phosphate moieties (e.g., phosphorothioate backbones). In general and unless otherwise specified, an analogue of a particular nucleotide has the same base-pairing specificity; i.e., an analogue of A will base-pair with T.

As used herein, the terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

As used herein, “CRISPR” refers to clustered regularly interspaced short palindromic repeats or any of the DNA loci that serve to direct CRISPR-associated proteins or similar nucleotide-directed nucleases. It also describes man-made, constructed, or selected systems derived using these frameworks or proteins. CRISPR systems and the related proteins vary among the currently described type I, type II and type III systems, though it is possible other analogous systems have yet to be described.

As used herein, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and other sequences and transcripts from a CRISPR locus. One or more tracr mate sequences operably linked to a guide sequence (e.g., direct repeat-spacer-direct repeat) can also be referred to as precrRNA (pre-CRISPR RNA) before processing or crRNA after processing by a nuclease. CRISPR systems can also include modified, swapped or engineered, guide, tracr or chimeric RNA sequences and the protein to which they interact (For example, Briner, et al., Mal Cell 56(2)333-9 (2014)). The methods disclosed herein may also be applicable to other, non-CRISPR nucleotide-directed nucleases.

As used herein, the term “guide sequence” refers to the portion of, for example, a guide RNA (gRNA) or single guide RNA (sgRNA) that confers the specificity of the nucleic acid-guide nuclease to its target, and that mediates the formation of the RNA-DNA duplex between the targeting RNA and the target DNA sequence. For example, the targeting specificity of a CRISPR-Cas9 complex is determined by the approximately 20-nt sequence at the 5′ end of the gRNA. The length of a guide sequence is typically between 17-24 bp. As used herein, “center of the guide sequence” refers to the midpoint of the guide sequence. For example, if a guide sequence were 20 nucleotides in length, the “center of the guide sequence” would be location between nucleotides 10 and 11, numbering from the 5′ end of the guide sequence to the 3′ end of the guide sequence.

As used herein, the terms “cleavage” or “cleaving” of nucleic acids, refer to the breakage of the covalent backbone of a nucleic acid molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered “sticky” ends. In certain embodiments cleavage refers to the double-stranded cleavage between nucleic acids within a double-stranded DNA or RNA chain.

As used herein, the terms “genomic region” or “genomic segment”, as used interchangeably herein, denote a contiguous length of nucleotides in a genome of an organism. A genomic region may be of a length as small as a few kb (e.g., at least 5 kb, at least 10 kb or at least 20 kb), up to an entire chromosome or more.

In some cases nucleotide sequences are provided using character representations recommended by the International Union of Pure and Applied Chemistry (IUPAC) or a subset thereof. IUPAC nucleotide codes used herein include, A=Adenine, C=Cytosine, G=Guanine, T=Thymine, U=Uracil, R=A or G, Y=C or T, S=G or C, W=A or T, K=G or T, M=A or C, B=C or G or T, D=A or G or T, H=A or C or T, V=A or C or G, N=any base, “.” or “-”=gap. In some embodiments the set {A, C, G, T, U} for adenosine, cytidine, guanosine, thymidine, and uridine respectively. In some embodiments the set {A, C, G, T, U, I, X, W} for adenosine, cytidine, guanosine, thymidine, uridine, inosine, uridine, xanthosine, pseudouridine respectively. In some embodiments the set of characters is {A, C, G, T, U, I, X, P, R, Y, N} for adenosine, cytidine, guanosine, thymidine, uridine, inosine, uridine, xanthosine, pseudouridine, unspecified purine, unspecified pyrimidine, and unspecified nucleotide respectively. The modified sequences, non-natural sequences, or sequences with modified binding, may be in the genomic, the guide or the tracr sequences.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or MEGALIGN (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Yl00, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. Mismatches can be similarly defined as differences between the natural binding partners of nucleotides. The number, position and type of mismatches can be calculated and used for identification or ranking purposes.

As used herein, “mutation” encompasses any change in a DNA, RNA, or protein sequence from the wild type sequence or some other reference, including without limitation point mutations, transitions, insertions, transversions, translocations, deletions, inversions, duplications, recombinations, or combinations thereof.

As used herein, in the context of a polynucleotide sequence that has undergone cleavage by an RNA-guided nuclease, the term “insertion” is used when the polynucleotide sequence has one or more extra bases compared with the polynucleotide sequence before cleavage by the RNA-guided nuclease occurred. Similarly, in the context of a polynucleotide sequence that has undergone cleavage by an RNA-guided nuclease, the term “deletion” is used when the polynucleotide sequence has one or more missing bases compared with the polynucleotide sequence before cleavage by the RNA-guided nuclease occurred. In the context of a polynucleotide sequence that has undergone cleavage by an RNA-guided nuclease, the term “indels” indicates either insertions or deletions. Cleavage by an RNA-guided nuclease can result in multiple indels, multiple insertions, multiple deletions or combinations of insertions of one or more nucleotides and deletions of one or more nucleotides.

### CRISPR

CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). By transfecting a cell with the required elements including a cas gene and specifically designed CRISPRs, a polynucleotide sequence can be cut and modified at virtually any desired location by unique targeting by, for example, a guide RNA that confers specificity to the nuclease. A number of methods exist for expressing the guide strand or Cas protein, including inducible expression of one or both. A number of methods exist for introducing the guide strand and Cas protein into cells including viral transduction, injection or micro-injection, nano-particle or other delivery, uptake of proteins, uptake of RNA or DNA, uptake of combination of protein and RNA or DNA. Combinations of methods can also be used, simultaneously or in sequence. Multiple rounds of delivery of RNA, DNA or protein can occur with or without further protein expression. Methods of preparing compositions for use in genome editing using the CRISPR/Cas systems are described in detail in WO 2013/176772 and WO 2014/018423, which are specifically incorporated by reference herein in their entireties.

### Nucleases

In some embodiments, the nuclease for use in the methods described herein is a Class 2 Cas nuclease. In some embodiments, the nuclease has double-strand endonuclease activity. In some embodiments, the nuclease comprises a Cas nuclease, such as a Class 2 Cas nuclease (which may be, e.g., a Cas nuclease of Type II, V, or VI). Class 2 Cas nucleases include, for example, Cas9, Cpf1, C2cl, C2c2, and C2c3 proteins and modifications thereof.

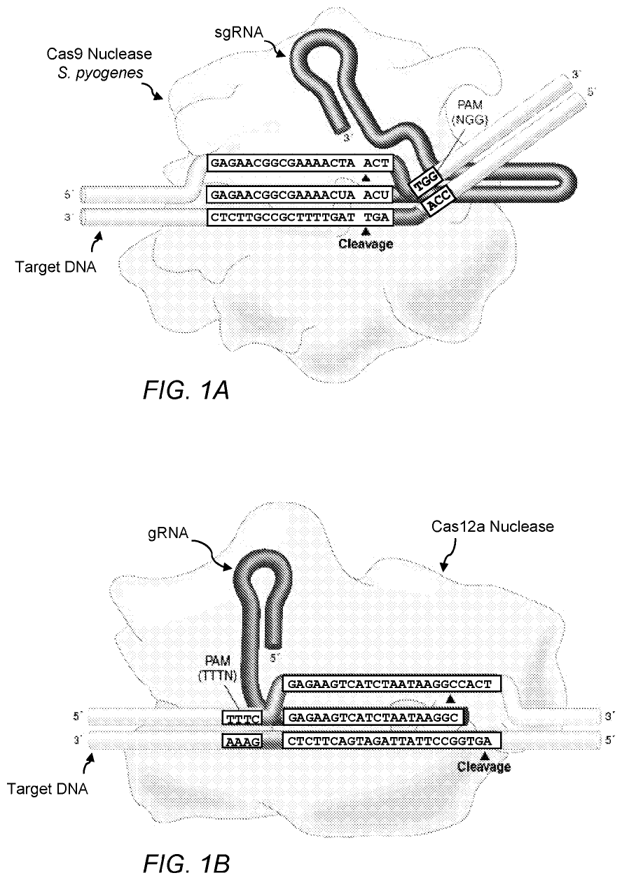

Examples of Cas9 nucleases include those of the type II CRISPR systems of S. pyogenes, S. aureus, and other prokaryotes (see, e.g., the list in the next paragraph), and modified (e.g., engineered or mutant) versions thereof. See, e.g., US2016/0312198 A1; US 2016/0312199 A1. FIG. 1A shows a Type II nuclease, featuring an sgRNA interacting with a target DNA sequence. PAM (NGG) sequence, guide sequence, target DNA sequence, and cleavage site are indicated. Other examples of Cas nucleases include a Csm or Cmr complex of a type III CRISPR system or the Cas 10, Csm1, or Cmr2 subunit thereof; and a Cascade complex of a type I CRISPR system, or the Cas3 subunit thereof. FIG. 1B shows a Type V nuclease, featuring a gRNA interacting with a target DNA sequence. PAM (TTTN) sequence, guide sequence, target DNA sequence, and cleavage site are indicated. In some embodiments, the Cas nuclease may be from a Type-IIA, Type-11B, or Type-IIC system. For discussion of various CRISPR systems and Cas nucleases see, e.g., Makarova et al., Nat. Rev. Microbiol. 9:467-477 (2011); Makarova et al., Nat. Rev. Microbiol, 13: 722-36 (2015); Shmakov et al., Molecular Cell, 60:385-397 (2015). In some embodiments, the RNA-guided DNA binding agent is a Cas nickase, e.g. a Cas9 nickase. In some embodiments, the RNA-guided DNA binding agent is an S. pyogenes Cas9 nuclease.

Non-limiting exemplary species that the nuclease (e.g., the Cas nuclease) can be derived from include but are not limited to Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter zari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In some embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus methanoplasma termitum, Eubacterium eligens, Moraxella bovoculi, Leptospira inadai, Porphyromonas crevioricanis, Prevotella disiens, or Porphyromonas macacae. In some embodiments, the Cas nuclease is a Cpf1 nuclease from an Acidaminococcus or Lachnospiraceae.

Wild type Cas9 has two nuclease domains: RuvC and HNH. The RuvC domain cleaves the non-target DNA strand, and the HNH domain cleaves the target strand of DNA. In some embodiments, the Cas9 nuclease comprises more than one RuvC domain and/or more than one HNH domain. In some embodiments, the Cas9 nuclease is a wild type Cas9. In some embodiments, the Cas9 is capable of inducing a double strand break in target DNA. In certain embodiments, the Cas nuclease can cleave one or both strands of dsDNA. In some embodiments, the Cas nuclease can cleave a single strand of DNA. In some embodiments, the Cas nuclease may not have DNA nickase activity.

In some embodiments, chimeric Cas nucleases are used, where one domain or region of the protein is replaced by a portion of a different protein. In some embodiments, a Cas nuclease domain may be replaced with a domain from a different nuclease such as Fok 1. In some embodiments, a Cas nuclease may be a modified nuclease, wherein the polypeptide sequence of the nuclease has been modified to confer, in some examples, advantageous properties to the nuclease. In some embodiments, the cleavage site of the nuclease relative to the location of the user-designated target sequence is unknown.

In other embodiments, the Cas nuclease may be from a Type-I CRISPR/Cas system. In some embodiments, the Cas nuclease may be a component of the Cascade complex of a Type-I CRISPR/Cas system In some embodiments, the Cas nuclease may be a Cas3 protein. In some embodiments, the Cas nuclease may be from a Type-III CRISPR/Cas system. In some embodiments, the Cas nuclease may have an RNA cleavage activity.

### Data Processing Pipeline

In some embodiments, disclosed herein is a data processing pipeline including the steps of receiving sample sequence data; applying quality control filters to the sample sequence data; aligning sequencing reads of the sample sequence data to a reference sequence; defining a window based on a sequence location within a nucleic acid guide and the locations of the ends of the nucleic acid guide sequence; and quantifying indels in the sample sequence data. In some embodiments, the plurality of sequencing reads is obtained from a next-generation sequencing (NGS) instrument. In some embodiments, the NGS sequencing instrument is an Illumina MiSeq™ machine. In some embodiments, sequencing and amplification adapters are trimmed by default, and so the returned NGS reads data do not have adapter sequences in the reads. In some embodiments, sequencing and amplification adapters are trimmed as a part of the data processing pipeline of the methods disclosed herein.

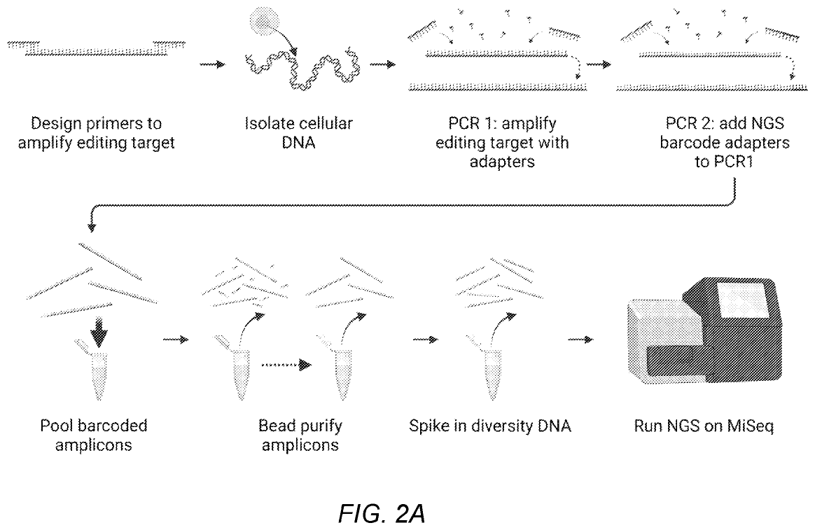

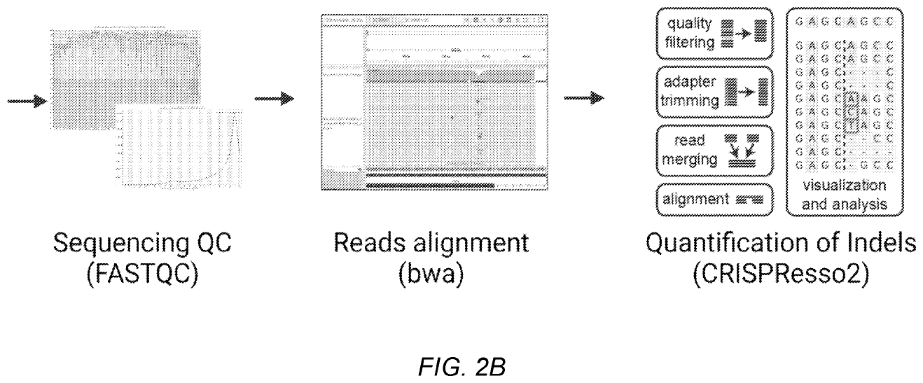

FIG. 2A shows steps of an upstream molecular biology workflow that can be performed in to generate data that is subsequently processed by the data processing pipeline disclosed herein. Steps of the upstream molecular biology workflow can include designing primers to amplify an editing target; isolating cellular DNA; PCR-amplifying the editing target with adapters; adding next-generation sequencing (NGS) adapters to PCR products; pooling barcoded amplicons; bead-purifying amplicons; spiking in diversity DNA; performing next-generation sequencing (NGS) on a sequencing instrument.

FIG. 2B shows steps of the computational pipeline disclosed herein. Steps can include quality control of received sequencing reads; alignment of sequencing reads to a target sequence; and quantification of indels according to the methods disclosed herein. These steps are described in further detail below.

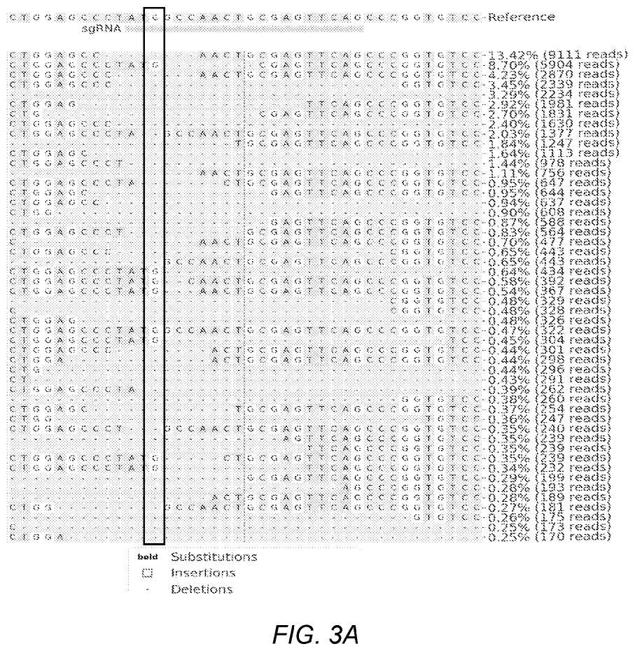

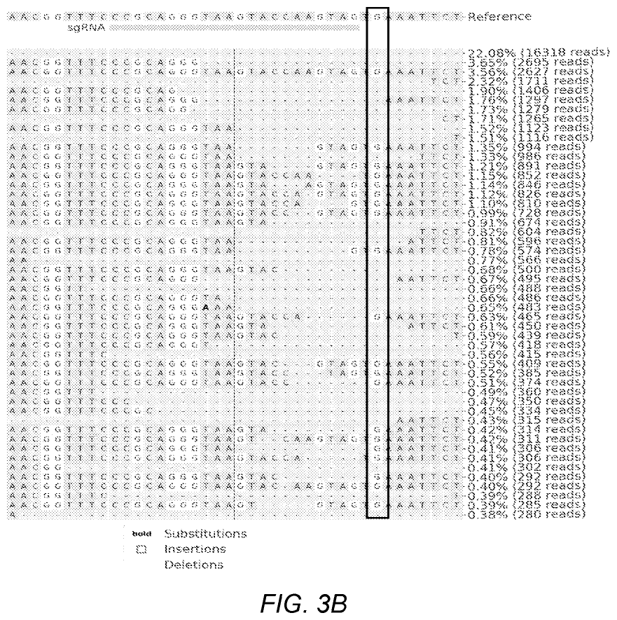

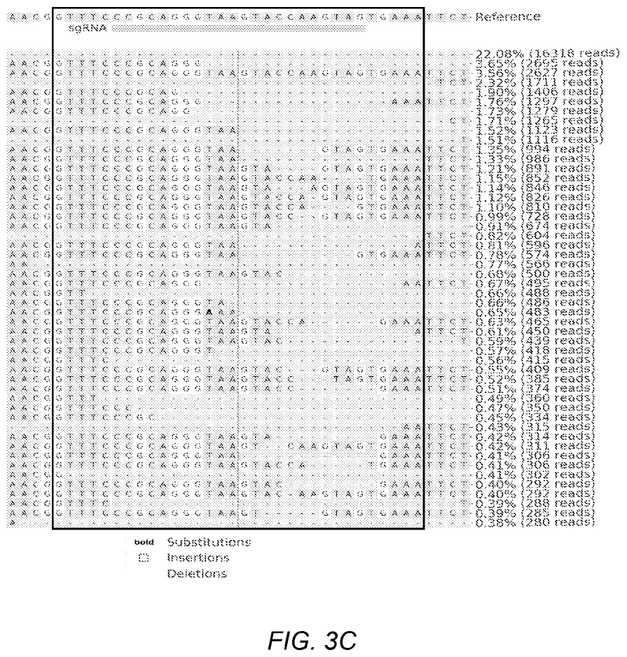

Previously available computational methods may underestimate the indel frequency in a target polynucleotide after cleavage by a nucleic acid-guided nuclease, for example, after cleavage with a Type II nuclease with a known cleavage site relative to the guide sequence. FIG. 3A is a schematic representation of received sequencing reads of nucleic-acid guided nuclease edited sample, comprising a plurality of sequencing reads, a subset of which contain indels as a result of cleavage by the nucleic-acid guided nuclease. The nuclease is Cas9, a Type II nuclease with a known cleavage site relative to the guide sequence. For each type of indel, the number of sequencing reads comprising that indel is indicated at right. The previously known cleavage site is indicated by a rectangular box.

Previously available computational methods may underestimate the indel frequency in a target polynucleotide after cleavage by a nucleic acid-guided nuclease, for example, after cleavage with a Type V nuclease with a known cleavage site relative to the guide sequence. FIG. 3B is a schematic representation of received sequencing reads of nucleic-acid guided nuclease edited sample, comprising a plurality of sequencing reads, a subset of which contain indels as a result of cleavage by the nucleic-acid guided nuclease. The nuclease is a novel Type V nuclease. For each type of indel, the number of sequencing reads comprising that indel is indicated at right. Several indels are missed by standard computational processing methods.

The computational pipeline disclosed herein and described in further detail below increases the accuracy of quantification of indel frequency in a target polynucleotide after cleavage by a nucleic acid-guided nuclease. FIG. 3C is a schematic representation of received sequencing reads of nucleic-acid guided nuclease edited sample, comprising a plurality of sequencing reads, a subset of which contain indels as a result of cleavage by the nucleic-acid guided nuclease. The nuclease is a novel Type V nuclease. For each type of indel, the number of sequencing reads comprising that indel is indicated at right. Custom data processing methods capture all indels generated by cleavage by the nuclease.

In some embodiments, the data processing pipeline disclosed herein includes one or more modules executed by the package of CRISPResso software tools (Clement K, et al. CRISPResso2 provides accurate and rapid genome editing sequence analysis. Nat Biotechnol. 2019 March; 37(3):224-226, and Canver M C, et al. Integrated design, execution, and analysis of arrayed and pooled CRISPR genome-editing experiments. Nat Protoc. 2018 May; 13(5):946-986, incorporated herein by reference in their entirety). In some embodiments, one or more read filtering parameters are applied to sample sequence data using CRISPResso in order to remove potentially false-positive indels from the sample sequence data, in order to improve the accuracy of the estimation of the frequency of indels in the sample sequence data. Read filtering parameters are described in further detail below. In some embodiments, read filtering is performed based on PHRED quality scores, which are described in Ewing B, Green P. Base-calling of automated sequencer traces using phred. II. Error probabilities. Genome Res. 1998 March; 8(3):186-94, which is incorporated by reference herein in its entirety. PHRED quality scores measure the quality of the identification of nucleotide base calls in sequencing reads generated by automated DNA sequence instruments.

In some embodiments, minimum average read quality (“q” or “min_average_read_quality”) is applied to filter sample sequence data, in order to remove potentially false-positive indels. This parameter allow for the specification of the minimum average quality score for inclusion of a read in subsequent analysis. The PHRED score represents the confidence in the assignment of a particular nucleotide in a read. The maximum score of 40 corresponds to an error rate of 0.01%. This average quality of a read is useful to filter out low-quality reads. In some embodiments, a “min_average_read_quality” value of 0, 5, 10, 15, 20, 25, 30, 35, or 40, is applied to sample sequence data.

In some embodiments, minimum single basepair score (“s” or “min_single_bp_quality”) is applied to filter sample sequence data, in order to remove potentially false-positive indels. This parameter allow for the specification of the minimum single-bp score for inclusion of a read in subsequent analysis. This parameter provides for more-stringent filtering; any read with a single-bp quality below the threshold will be discarded. In some embodiments, a “min_single_bp_quality” value of 0, 1, 2, 3, 4, 5, 6, 7, 8, 9, 10, 11, 12, 13, 14, 15, 16, 17, 18, 19, or 20, is applied to sample sequence data.

In some embodiments, an amplicon minimum alignment score (“amas” or “amplicon_min_alignment_score”) is applied to filter sample sequence data. After reads are aligned to a reference sequence, the homology is calculated as the number of basepairs they have in common. This is useful for filtering erroneous reads that do not align to the target sequence, for example arising from alternate primer locations. In some embodiments, a “amplicon_min_alignment_score” value of 0, 10, 20, 30, 40, 50, 60, 70, 80, 90, or 100, is applied to sample sequence data.

In some embodiments, a combination of a “min_average_read_quality” score, a “min_single_bp_quality” score, and a “amplicon_min_alignment_score” is applied to filter sample sequence data in order to improve the estimation of indel frequency in sample sequence data after cleavage by a target polynucleotide sequence by a nuclease. In some embodiments, a combination of a “min_average_read_quality” score, a “min_single_bp_quality” score, and a “amplicon_min_alignment_score,” further in combination with a window size based on a sequence location within a guide sequence, is applied to filter sample sequence data in order to improve the estimation of indel frequency in sample sequence data after cleavage by a target polynucleotide sequence by a nuclease. As described in Table 1 below, combinations of these parameters may be used to enhance the accuracy of indel quantification according to the methods disclosed herein.

In some embodiments, a combination of a “min_average_read_quality” score, a “min_single_bp_quality” score, and a “amplicon_min_alignment_score” is applied to filter sample sequence data in order to improve the estimation of indel frequency in sample sequence data after cleavage by a target polynucleotide sequence by a nuclease. In some embodiments, a combination of a “min_average_read_quality” score, a “min_single_bp_quality” score, and a “amplicon_min_alignment_score,” further in combination with a window size based on a sequence location of a known insertion site, is applied to filter sample sequence data in order to improve the estimation of indel frequency in sample sequence data after cleavage by a target polynucleotide sequence by a nuclease and insertion of a polynucleotide sequence. As described in Table 1 below, combinations of these parameters may be used to enhance the accuracy of indel quantification according to the methods disclosed herein.

In some embodiments, if the sample sequence data include paired-end reads, the data processing pipeline disclosed herein includes one or modules executed by the package of FLASH software tools (Magoč T, Salzberg S L. FLASH: fast length adjustment of short reads to improve genome assemblies. Bioinformatics. 2011 Nov. 1; 27(21):2957-63, incorporated by reference herein in its entirety). FLASH is a rapid and accurate software tool to merge paired-end reads from next-generation sequencing experiments, designed to merge pairs of reads when the original DNA fragments are shorter than twice the length of reads. The resulting longer reads can significantly improve genome assemblies. FLASH calculates a mismatch ratio within two overlapped regions. If the mismatch ratio is over a threshold mismatch ratio, FLASH determines that the reads are an incorrect overlap. In some embodiments, paired-end reads are merged using FLASH in order to produce single reads for alignment to the target reference sequence and reduces sequencing errors that may be present at the end of sequencing reads.

In some embodiments, a maximum paired-end reads overlap (“max_paired_end_reads_overlap”) is applied to paired-end sample sequence data for the FLASH read merging step of the data processing pipeline. This parameter represents the maximum overlap length expected in approximately 90% of read pairs. In some embodiments, a “max_paired_end_reads_overlap” value of 50, 60, 70, 80, 90, 100, 110, 120, 130, 140, 150, 160, 170, 180, 190, 200, 210, 220, 230, 240, 250, 260, 270, 280, 290, or 300 is applied to sample sequence data.

In some embodiments, a minimum paired-end reads overlap (“min_paired_end_reads_overlap”) is applied to paired-end sample sequence data for the FLASH read merging step of the data processing pipeline. This parameter represents the minimum required overlap length between two reads to provide a confident overlap. In some embodiments, a “max paired end reads overlap” value of 0, 10, 20, 30, 40, 50, 60, 70, 80, 90, or 100, is applied to sample sequence data.

In some embodiments, a combination of a “min_average_read_quality” score, a “min_single_bp_quality” score, a “min_paired_end_reads_overlap,” and a “max_paired_end_reads_overlap” is applied to filter sample sequence data in order to improve the estimation of indel frequency in sample sequence data after cleavage by a target polynucleotide sequence by a nuclease. In some embodiments, a combination of a “min_average_read_quality” score, a “min_single_bp_quality” score, a “min_paired_end_reads_overlap,” and a “max_paired_end_reads_overlap” further in combination with a window size based on a sequence location within a guide sequence, is applied to filter sample sequence data in order to improve the estimation of indel frequency in sample sequence data after cleavage by a target polynucleotide sequence by a nuclease. As described in Table 1 below, combinations of these parameters may be used to enhance the accuracy of indel quantification according to the methods disclosed herein.

In some embodiments, a combination of a “min_average_read_quality” score, a “min_single_bp_quality” score, a “min_paired_end_reads_overlap,” and a “max_paired_end_reads_overlap” is applied to filter sample sequence data in order to improve the estimation of indel frequency in sample sequence data after cleavage by a target polynucleotide sequence by a nuclease. In some embodiments, a combination of a “min_average_read_quality” score, a “min_single_bp_quality” score, a “min_paired_end_reads_overlap,” and a “max_paired_end_reads_overlap” further in combination with a window size based on a sequence location of a known insertion site, is applied to filter sample sequence data in order to improve the estimation of indel frequency in sample sequence data after cleavage by a target polynucleotide sequence by a nuclease and insertion of a polynucleotide sequence. As described in Table 1 below, combinations of these parameters may be used to enhance the accuracy of indel quantification according to the methods disclosed herein.

In some embodiments, the quantification window extends 50, 40, 30, 20, 10, or 5 nucleotides 5′ to the 5′ end of the guide sequence. In some embodiments, the quantification window extends 50, 40, 30, 20, 10, or 5 nucleotides 3′ to the 3′ end of the guide sequence. In some embodiments, the quantification window extends 50, 40, 30, 20, 10, or 5 nucleotides 5′ to a known insertion site. In some embodiments, the quantification window extends 50, 40, 30, 20, 10, or 5 nucleotides 3′ to a known insertion site. In some embodiments, the quantification window is 10, 20, 30, 40, 50, 60, 70, 80, 90, 100, 200, 300, 400, or 500 nucleotides in length.

In some embodiments, the indel to be detected is about 1, 2, 3, 4, 5, 6, 7, 8, 9, 10 or more nucleotides in length. In some embodiments, the indel to be detected is about 10, 20, 30, 40, 50, 60, 70, 80, 90, 100 or more nucleotides in length. In some embodiments, the indel comprises a CRISPR-mediated donor insertion. In some embodiments, the indel is the result of homology directed repair (HDR). In some embodiments, the indel is the result of insertion by a CRISPR-associated transposase (CAST). In some embodiments, the insertion comprises sequences from a genomic library.

TABLE 1

| | | | | | window |
|---|---|---|---|---|---|
| | Min | Max | | | size |
| | paired | paired | Min | Min | from end |
| Condition | end reads | end reads | single bp | average | of guide |
| Set | overlap | overlap | quality | read quality | sequence) |
| 1 | 0 | 100 | 0 | 0 | 0 |
| 2 | 0 | 250 | 0 | 0 | 0 |
| 3 | 5 | 100 | 0 | 0 | 0 |
| 4 | 5 | 250 | 0 | 0 | 0 |
| 5 | 10 | 100 | 0 | 0 | 0 |
| 6 | 10 | 250 | 0 | 0 | 0 |
| 7 | 15 | 100 | 0 | 0 | 0 |
| 8 | 15 | 250 | 0 | 0 | 0 |
| 9 | 0 | 100 | 10 | 0 | 0 |
| 10 | 0 | 250 | 10 | 0 | 0 |
| 11 | 5 | 100 | 10 | 0 | 0 |
| 12 | 5 | 250 | 10 | 0 | 0 |
| 13 | 10 | 100 | 10 | 0 | 0 |
| 14 | 10 | 250 | 10 | 0 | 0 |
| 15 | 15 | 100 | 10 | 0 | 0 |
| 16 | 15 | 250 | 10 | 0 | 0 |
| 17 | 0 | 100 | 20 | 0 | 0 |
| 18 | 0 | 250 | 20 | 0 | 0 |
| 19 | 5 | 100 | 20 | 0 | 0 |
| 20 | 5 | 250 | 20 | 0 | 0 |
| 21 | 10 | 100 | 20 | 0 | 0 |
| 22 | 10 | 250 | 20 | 0 | 0 |
| 23 | 15 | 100 | 20 | 0 | 0 |
| 24 | 15 | 250 | 20 | 0 | 0 |
| 25 | 0 | 100 | 30 | 0 | 0 |
| 26 | 0 | 250 | 30 | 0 | 0 |
| 27 | 5 | 100 | 30 | 0 | 0 |
| 28 | 5 | 250 | 30 | 0 | 0 |
| 29 | 10 | 100 | 30 | 0 | 0 |
| 30 | 10 | 250 | 30 | 0 | 0 |
| 31 | 15 | 100 | 30 | 0 | 0 |
| 32 | 15 | 250 | 30 | 0 | 0 |
| 33 | 0 | 100 | 0 | 20 | 0 |
| 34 | 0 | 250 | 0 | 20 | 0 |
| 35 | 5 | 100 | 0 | 20 | 0 |
| 36 | 5 | 250 | 0 | 20 | 0 |
| 37 | 10 | 100 | 0 | 20 | 0 |
| 38 | 10 | 250 | 0 | 20 | 0 |
| 39 | 15 | 100 | 0 | 20 | 0 |
| 40 | 15 | 250 | 0 | 20 | 0 |
| 41 | 0 | 100 | 10 | 20 | 0 |
| 42 | 0 | 250 | 10 | 20 | 0 |
| 43 | 5 | 100 | 10 | 20 | 0 |
| 44 | 5 | 250 | 10 | 20 | 0 |
| 45 | 10 | 100 | 10 | 20 | 0 |
| 46 | 10 | 250 | 10 | 20 | 0 |
| 47 | 15 | 100 | 10 | 20 | 0 |
| 48 | 15 | 250 | 10 | 20 | 0 |
| 49 | 0 | 100 | 20 | 20 | 0 |
| 50 | 0 | 250 | 20 | 20 | 0 |
| 51 | 5 | 100 | 20 | 20 | 0 |
| 52 | 5 | 250 | 20 | 20 | 0 |
| 53 | 10 | 100 | 20 | 20 | 0 |
| 54 | 10 | 250 | 20 | 20 | 0 |
| 55 | 15 | 100 | 20 | 20 | 0 |
| 56 | 15 | 250 | 20 | 20 | 0 |
| 57 | 0 | 100 | 30 | 20 | 0 |
| 58 | 0 | 250 | 30 | 20 | 0 |
| 59 | 5 | 100 | 30 | 20 | 0 |
| 60 | 5 | 250 | 30 | 20 | 0 |
| 61 | 10 | 100 | 30 | 20 | 0 |
| 62 | 10 | 250 | 30 | 20 | 0 |
| 63 | 15 | 100 | 30 | 20 | 0 |
| 64 | 15 | 250 | 30 | 20 | 0 |
| 65 | 0 | 100 | 0 | 30 | 0 |
| 66 | 0 | 250 | 0 | 30 | 0 |
| 67 | 5 | 100 | 0 | 30 | 0 |
| 68 | 5 | 250 | 0 | 30 | 0 |
| 69 | 10 | 100 | 0 | 30 | 0 |
| 70 | 10 | 250 | 0 | 30 | 0 |
| 71 | 15 | 100 | 0 | 30 | 0 |
| 72 | 15 | 250 | 0 | 30 | 0 |
| 73 | 0 | 100 | 10 | 30 | 0 |
| 74 | 0 | 250 | 10 | 30 | 0 |
| 75 | 5 | 100 | 10 | 30 | 0 |
| 76 | 5 | 250 | 10 | 30 | 0 |
| 77 | 10 | 100 | 10 | 30 | 0 |
| 78 | 10 | 250 | 10 | 30 | 0 |
| 79 | 15 | 100 | 10 | 30 | 0 |
| 80 | 15 | 250 | 10 | 30 | 0 |
| 81 | 0 | 100 | 20 | 30 | 0 |
| 82 | 0 | 250 | 20 | 30 | 0 |
| 83 | 5 | 100 | 20 | 30 | 0 |
| 84 | 5 | 250 | 20 | 30 | 0 |
| 85 | 10 | 100 | 20 | 30 | 0 |
| 86 | 10 | 250 | 20 | 30 | 0 |
| 87 | 15 | 100 | 20 | 30 | 0 |
| 88 | 15 | 250 | 20 | 30 | 0 |
| 89 | 0 | 100 | 30 | 30 | 0 |
| 90 | 0 | 250 | 30 | 30 | 0 |
| 91 | 5 | 100 | 30 | 30 | 0 |
| 92 | 5 | 250 | 30 | 30 | 0 |
| 93 | 10 | 100 | 30 | 30 | 0 |
| 94 | 10 | 250 | 30 | 30 | 0 |
| 95 | 15 | 100 | 30 | 30 | 0 |
| 96 | 15 | 250 | 30 | 30 | 0 |
| 97 | 0 | 100 | 0 | 40 | 0 |
| 98 | 0 | 250 | 0 | 40 | 0 |
| 99 | 5 | 100 | 0 | 40 | 0 |
| 100 | 5 | 250 | 0 | 40 | 0 |
| 101 | 10 | 100 | 0 | 40 | 0 |
| 102 | 10 | 250 | 0 | 40 | 0 |
| 103 | 15 | 100 | 0 | 40 | 0 |
| 104 | 15 | 250 | 0 | 40 | 0 |
| 105 | 0 | 100 | 10 | 40 | 0 |
| 106 | 0 | 250 | 10 | 40 | 0 |
| 107 | 5 | 100 | 10 | 40 | 0 |
| 108 | 5 | 250 | 10 | 40 | 0 |
| 109 | 10 | 100 | 10 | 40 | 0 |
| 110 | 10 | 250 | 10 | 40 | 0 |
| 111 | 15 | 100 | 10 | 40 | 0 |
| 112 | 15 | 250 | 10 | 40 | 0 |
| 113 | 0 | 100 | 20 | 40 | 0 |
| 114 | 0 | 250 | 20 | 40 | 0 |
| 115 | 5 | 100 | 20 | 40 | 0 |
| 116 | 5 | 250 | 20 | 40 | 0 |
| 117 | 10 | 100 | 20 | 40 | 0 |
| 118 | 10 | 250 | 20 | 40 | 0 |
| 119 | 15 | 100 | 20 | 40 | 0 |
| 120 | 15 | 250 | 20 | 40 | 0 |
| 121 | 0 | 100 | 30 | 40 | 0 |
| 122 | 0 | 250 | 30 | 40 | 0 |
| 123 | 5 | 100 | 30 | 40 | 0 |
| 124 | 5 | 250 | 30 | 40 | 0 |
| 125 | 10 | 100 | 30 | 40 | 0 |
| 126 | 10 | 250 | 30 | 40 | 0 |
| 127 | 15 | 100 | 30 | 40 | 0 |
| 128 | 15 | 250 | 30 | 40 | 0 |
| 129 | 0 | 100 | 0 | 0 | 5 |
| 130 | 0 | 250 | 0 | 0 | 5 |
| 131 | 5 | 100 | 0 | 0 | 5 |
| 132 | 5 | 250 | 0 | 0 | 5 |
| 133 | 10 | 100 | 0 | 0 | 5 |
| 134 | 10 | 250 | 0 | 0 | 5 |
| 135 | 15 | 100 | 0 | 0 | 5 |
| 136 | 15 | 250 | 0 | 0 | 5 |
| 137 | 0 | 100 | 10 | 0 | 5 |
| 138 | 0 | 250 | 10 | 0 | 5 |
| 139 | 5 | 100 | 10 | 0 | 5 |
| 140 | 5 | 250 | 10 | 0 | 5 |
| 141 | 10 | 100 | 10 | 0 | 5 |
| 142 | 10 | 250 | 10 | 0 | 5 |
| 143 | 15 | 100 | 10 | 0 | 5 |
| 144 | 15 | 250 | 10 | 0 | 5 |
| 145 | 0 | 100 | 20 | 0 | 5 |
| 146 | 0 | 250 | 20 | 0 | 5 |
| 147 | 5 | 100 | 20 | 0 | 5 |
| 148 | 5 | 250 | 20 | 0 | 5 |
| 149 | 10 | 100 | 20 | 0 | 5 |
| 150 | 10 | 250 | 20 | 0 | 5 |
| 151 | 15 | 100 | 20 | 0 | 5 |
| 152 | 15 | 250 | 20 | 0 | 5 |
| 153 | 0 | 100 | 30 | 0 | 5 |
| 154 | 0 | 250 | 30 | 0 | 5 |
| 155 | 5 | 100 | 30 | 0 | 5 |
| 156 | 5 | 250 | 30 | 0 | 5 |
| 157 | 10 | 100 | 30 | 0 | 5 |
| 158 | 10 | 250 | 30 | 0 | 5 |
| 159 | 15 | 100 | 30 | 0 | 5 |
| 160 | 15 | 250 | 30 | 0 | 5 |
| 161 | 0 | 100 | 0 | 20 | 5 |
| 162 | 0 | 250 | 0 | 20 | 5 |
| 163 | 5 | 100 | 0 | 20 | 5 |
| 164 | 5 | 250 | 0 | 20 | 5 |
| 165 | 10 | 100 | 0 | 20 | 5 |
| 166 | 10 | 250 | 0 | 20 | 5 |
| 167 | 15 | 100 | 0 | 20 | 5 |
| 168 | 15 | 250 | 0 | 20 | 5 |
| 169 | 0 | 100 | 10 | 20 | 5 |
| 170 | 0 | 250 | 10 | 20 | 5 |
| 171 | 5 | 100 | 10 | 20 | 5 |
| 172 | 5 | 250 | 10 | 20 | 5 |
| 173 | 10 | 100 | 10 | 20 | 5 |
| 174 | 10 | 250 | 10 | 20 | 5 |
| 175 | 15 | 100 | 10 | 20 | 5 |
| 176 | 15 | 250 | 10 | 20 | 5 |
| 177 | 0 | 100 | 20 | 20 | 5 |
| 178 | 0 | 250 | 20 | 20 | 5 |
| 179 | 5 | 100 | 20 | 20 | 5 |
| 180 | 5 | 250 | 20 | 20 | 5 |
| 181 | 10 | 100 | 20 | 20 | 5 |
| 182 | 10 | 250 | 20 | 20 | 5 |
| 183 | 15 | 100 | 20 | 20 | 5 |
| 184 | 15 | 250 | 20 | 20 | 5 |
| 185 | 0 | 100 | 30 | 20 | 5 |
| 186 | 0 | 250 | 30 | 20 | 5 |
| 187 | 5 | 100 | 30 | 20 | 5 |
| 188 | 5 | 250 | 30 | 20 | 5 |
| 189 | 10 | 100 | 30 | 20 | 5 |
| 190 | 10 | 250 | 30 | 20 | 5 |
| 191 | 15 | 100 | 30 | 20 | 5 |
| 192 | 15 | 250 | 30 | 20 | 5 |
| 193 | 0 | 100 | 0 | 30 | 5 |
| 194 | 0 | 250 | 0 | 30 | 5 |
| 195 | 5 | 100 | 0 | 30 | 5 |
| 196 | 5 | 250 | 0 | 30 | 5 |
| 197 | 10 | 100 | 0 | 30 | 5 |
| 198 | 10 | 250 | 0 | 30 | 5 |
| 199 | 15 | 100 | 0 | 30 | 5 |
| 200 | 15 | 250 | 0 | 30 | 5 |
| 201 | 0 | 100 | 10 | 30 | 5 |
| 202 | 0 | 250 | 10 | 30 | 5 |
| 203 | 5 | 100 | 10 | 30 | 5 |
| 204 | 5 | 250 | 10 | 30 | 5 |
| 205 | 10 | 100 | 10 | 30 | 5 |
| 206 | 10 | 250 | 10 | 30 | 5 |
| 207 | 15 | 100 | 10 | 30 | 5 |
| 208 | 15 | 250 | 10 | 30 | 5 |
| 209 | 0 | 100 | 20 | 30 | 5 |
| 210 | 0 | 250 | 20 | 30 | 5 |
| 211 | 5 | 100 | 20 | 30 | 5 |
| 212 | 5 | 250 | 20 | 30 | 5 |
| 213 | 10 | 100 | 20 | 30 | 5 |
| 214 | 10 | 250 | 20 | 30 | 5 |
| 215 | 15 | 100 | 20 | 30 | 5 |
| 216 | 15 | 250 | 20 | 30 | 5 |
| 217 | 0 | 100 | 30 | 30 | 5 |
| 218 | 0 | 250 | 30 | 30 | 5 |
| 219 | 5 | 100 | 30 | 30 | 5 |
| 220 | 5 | 250 | 30 | 30 | 5 |
| 221 | 10 | 100 | 30 | 30 | 5 |
| 222 | 10 | 250 | 30 | 30 | 5 |
| 223 | 15 | 100 | 30 | 30 | 5 |
| 224 | 15 | 250 | 30 | 30 | 5 |
| 225 | 0 | 100 | 0 | 40 | 5 |
| 226 | 0 | 250 | 0 | 40 | 5 |
| 227 | 5 | 100 | 0 | 40 | 5 |
| 228 | 5 | 250 | 0 | 40 | 5 |
| 229 | 10 | 100 | 0 | 40 | 5 |
| 230 | 10 | 250 | 0 | 40 | 5 |
| 231 | 15 | 100 | 0 | 40 | 5 |
| 232 | 15 | 250 | 0 | 40 | 5 |
| 233 | 0 | 100 | 10 | 40 | 5 |
| 234 | 0 | 250 | 10 | 40 | 5 |
| 235 | 5 | 100 | 10 | 40 | 5 |
| 236 | 5 | 250 | 10 | 40 | 5 |
| 237 | 10 | 100 | 10 | 40 | 5 |
| 238 | 10 | 250 | 10 | 40 | 5 |
| 239 | 15 | 100 | 10 | 40 | 5 |
| 240 | 15 | 250 | 10 | 40 | 5 |
| 241 | 0 | 100 | 20 | 40 | 5 |
| 242 | 0 | 250 | 20 | 40 | 5 |
| 243 | 5 | 100 | 20 | 40 | 5 |
| 244 | 5 | 250 | 20 | 40 | 5 |
| 245 | 10 | 100 | 20 | 40 | 5 |
| 246 | 10 | 250 | 20 | 40 | 5 |
| 247 | 15 | 100 | 20 | 40 | 5 |
| 248 | 15 | 250 | 20 | 40 | 5 |
| 249 | 0 | 100 | 30 | 40 | 5 |
| 250 | 0 | 250 | 30 | 40 | 5 |
| 251 | 5 | 100 | 30 | 40 | 5 |
| 252 | 5 | 250 | 30 | 40 | 5 |
| 253 | 10 | 100 | 30 | 40 | 5 |
| 254 | 10 | 250 | 30 | 40 | 5 |
| 255 | 15 | 100 | 30 | 40 | 5 |
| 256 | 15 | 250 | 30 | 40 | 5 |
| 257 | 0 | 100 | 0 | 0 | 10 |
| 258 | 0 | 250 | 0 | 0 | 10 |
| 259 | 5 | 100 | 0 | 0 | 10 |
| 260 | 5 | 250 | 0 | 0 | 10 |
| 261 | 10 | 100 | 0 | 0 | 10 |
| 262 | 10 | 250 | 0 | 0 | 10 |
| 263 | 15 | 100 | 0 | 0 | 10 |
| 264 | 15 | 250 | 0 | 0 | 10 |
| 265 | 0 | 100 | 10 | 0 | 10 |
| 266 | 0 | 250 | 10 | 0 | 10 |
| 267 | 5 | 100 | 10 | 0 | 10 |
| 268 | 5 | 250 | 10 | 0 | 10 |
| 269 | 10 | 100 | 10 | 0 | 10 |
| 270 | 10 | 250 | 10 | 0 | 10 |
| 271 | 15 | 100 | 10 | 0 | 10 |
| 272 | 15 | 250 | 10 | 0 | 10 |
| 273 | 0 | 100 | 20 | 0 | 10 |
| 274 | 0 | 250 | 20 | 0 | 10 |
| 275 | 5 | 100 | 20 | 0 | 10 |
| 276 | 5 | 250 | 20 | 0 | 10 |
| 277 | 10 | 100 | 20 | 0 | 10 |
| 278 | 10 | 250 | 20 | 0 | 10 |
| 279 | 15 | 100 | 20 | 0 | 10 |
| 280 | 15 | 250 | 20 | 0 | 10 |
| 281 | 0 | 100 | 30 | 0 | 10 |
| 282 | 0 | 250 | 30 | 0 | 10 |
| 283 | 5 | 100 | 30 | 0 | 10 |
| 284 | 5 | 250 | 30 | 0 | 10 |
| 285 | 10 | 100 | 30 | 0 | 10 |
| 286 | 10 | 250 | 30 | 0 | 10 |
| 287 | 15 | 100 | 30 | 0 | 10 |
| 288 | 15 | 250 | 30 | 0 | 10 |
| 289 | 0 | 100 | 0 | 20 | 10 |
| 290 | 0 | 250 | 0 | 20 | 10 |
| 291 | 5 | 100 | 0 | 20 | 10 |
| 292 | 5 | 250 | 0 | 20 | 10 |
| 293 | 10 | 100 | 0 | 20 | 10 |
| 294 | 10 | 250 | 0 | 20 | 10 |
| 295 | 15 | 100 | 0 | 20 | 10 |
| 296 | 15 | 250 | 0 | 20 | 10 |
| 297 | 0 | 100 | 10 | 20 | 10 |
| 298 | 0 | 250 | 10 | 20 | 10 |
| 299 | 5 | 100 | 10 | 20 | 10 |
| 300 | 5 | 250 | 10 | 20 | 10 |
| 301 | 10 | 100 | 10 | 20 | 10 |
| 302 | 10 | 250 | 10 | 20 | 10 |
| 303 | 15 | 100 | 10 | 20 | 10 |
| 304 | 15 | 250 | 10 | 20 | 10 |
| 305 | 0 | 100 | 20 | 20 | 10 |
| 306 | 0 | 250 | 20 | 20 | 10 |
| 307 | 5 | 100 | 20 | 20 | 10 |
| 308 | 5 | 250 | 20 | 20 | 10 |
| 309 | 10 | 100 | 20 | 20 | 10 |
| 310 | 10 | 250 | 20 | 20 | 10 |
| 311 | 15 | 100 | 20 | 20 | 10 |
| 312 | 15 | 250 | 20 | 20 | 10 |
| 313 | 0 | 100 | 30 | 20 | 10 |
| 314 | 0 | 250 | 30 | 20 | 10 |
| 315 | 5 | 100 | 30 | 20 | 10 |
| 316 | 5 | 250 | 30 | 20 | 10 |
| 317 | 10 | 100 | 30 | 20 | 10 |
| 318 | 10 | 250 | 30 | 20 | 10 |
| 319 | 15 | 100 | 30 | 20 | 10 |
| 320 | 15 | 250 | 30 | 20 | 10 |
| 321 | 0 | 100 | 0 | 30 | 10 |
| 322 | 0 | 250 | 0 | 30 | 10 |
| 323 | 5 | 100 | 0 | 30 | 10 |
| 324 | 5 | 250 | 0 | 30 | 10 |
| 325 | 10 | 100 | 0 | 30 | 10 |
| 326 | 10 | 250 | 0 | 30 | 10 |
| 327 | 15 | 100 | 0 | 30 | 10 |
| 328 | 15 | 250 | 0 | 30 | 10 |
| 329 | 0 | 100 | 10 | 30 | 10 |
| 330 | 0 | 250 | 10 | 30 | 10 |
| 331 | 5 | 100 | 10 | 30 | 10 |
| 332 | 5 | 250 | 10 | 30 | 10 |
| 333 | 10 | 100 | 10 | 30 | 10 |
| 334 | 10 | 250 | 10 | 30 | 10 |
| 335 | 15 | 100 | 10 | 30 | 10 |
| 336 | 15 | 250 | 10 | 30 | 10 |
| 337 | 0 | 100 | 20 | 30 | 10 |
| 338 | 0 | 250 | 20 | 30 | 10 |
| 339 | 5 | 100 | 20 | 30 | 10 |
| 340 | 5 | 250 | 20 | 30 | 10 |
| 341 | 10 | 100 | 20 | 30 | 10 |
| 342 | 10 | 250 | 20 | 30 | 10 |
| 343 | 15 | 100 | 20 | 30 | 10 |
| 344 | 15 | 250 | 20 | 30 | 10 |
| 345 | 0 | 100 | 30 | 30 | 10 |
| 346 | 0 | 250 | 30 | 30 | 10 |
| 347 | 5 | 100 | 30 | 30 | 10 |
| 348 | 5 | 250 | 30 | 30 | 10 |
| 349 | 10 | 100 | 30 | 30 | 10 |
| 350 | 10 | 250 | 30 | 30 | 10 |
| 351 | 15 | 100 | 30 | 30 | 10 |
| 352 | 15 | 250 | 30 | 30 | 10 |
| 353 | 0 | 100 | 0 | 40 | 10 |
| 354 | 0 | 250 | 0 | 40 | 10 |
| 355 | 5 | 100 | 0 | 40 | 10 |
| 356 | 5 | 250 | 0 | 40 | 10 |
| 357 | 10 | 100 | 0 | 40 | 10 |
| 358 | 10 | 250 | 0 | 40 | 10 |
| 359 | 15 | 100 | 0 | 40 | 10 |
| 360 | 15 | 250 | 0 | 40 | 10 |
| 361 | 0 | 100 | 10 | 40 | 10 |
| 362 | 0 | 250 | 10 | 40 | 10 |
| 363 | 5 | 100 | 10 | 40 | 10 |
| 364 | 5 | 250 | 10 | 40 | 10 |
| 365 | 10 | 100 | 10 | 40 | 10 |
| 366 | 10 | 250 | 10 | 40 | 10 |
| 367 | 15 | 100 | 10 | 40 | 10 |
| 368 | 15 | 250 | 10 | 40 | 10 |
| 369 | 0 | 100 | 20 | 40 | 10 |
| 370 | 0 | 250 | 20 | 40 | 10 |
| 371 | 5 | 100 | 20 | 40 | 10 |
| 372 | 5 | 250 | 20 | 40 | 10 |
| 373 | 10 | 100 | 20 | 40 | 10 |
| 374 | 10 | 250 | 20 | 40 | 10 |
| 375 | 15 | 100 | 20 | 40 | 10 |
| 376 | 15 | 250 | 20 | 40 | 10 |
| 377 | 0 | 100 | 30 | 40 | 10 |
| 378 | 0 | 250 | 30 | 40 | 10 |
| 379 | 5 | 100 | 30 | 40 | 10 |
| 380 | 5 | 250 | 30 | 40 | 10 |
| 381 | 10 | 100 | 30 | 40 | 10 |
| 382 | 10 | 250 | 30 | 40 | 10 |
| 383 | 15 | 100 | 30 | 40 | 10 |
| 384 | 15 | 250 | 30 | 40 | 10 |

### Computer Implementation of the Methods

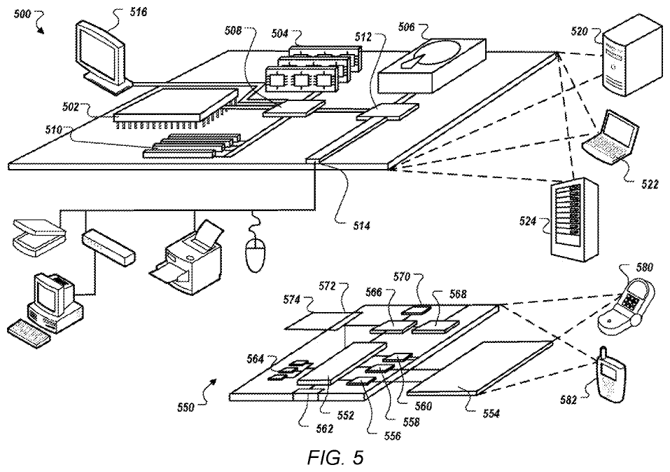

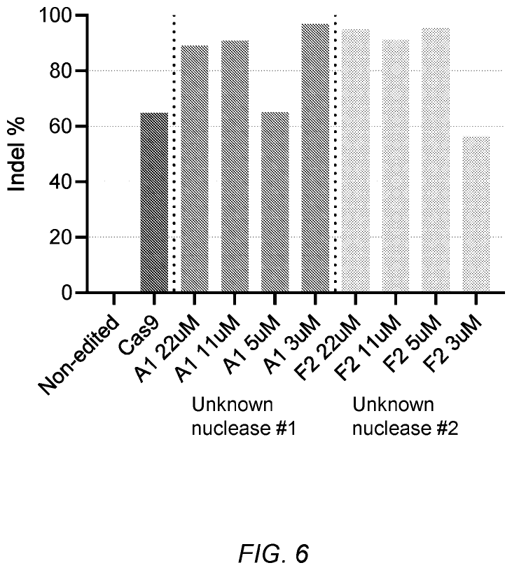

FIG. 5 is a diagram of computer system 500 components that can be used to implement a computational pipeline for indel quantification for polynucleotide sequences that have been cleaved by nucleic-acid guided nucleases, including uncharacterized nucleases. Computer system 500 can be used to implement methods that include parameters for alignment of next-generation sequencing (NGS) reads from targeted amplicon sequencing (TAS) of nuclease-edited or control samples that have been obtained and aligned to reference amplicon sequences.

Computing device 500 is intended to represent various forms of digital computers, such as laptops, desktops, workstations, personal digital assistants, servers, blade servers, mainframes, and other appropriate computers. Computing device 550 is intended to represent various forms of mobile devices, such as personal digital assistants, cellular telephones, smartphones, and other similar computing devices. Additionally, computing device 500 or 550 can include Universal Serial Bus (USB) flash drives. The USB flash drives can store operating systems and other applications. The USB flash drives can include input/output components, such as a wireless transmitter or USB connector that can be inserted into a USB port of another computing device. The components shown here, their connections and relationships, and their functions, are meant to be examples only, and are not meant to limit implementations of the methods and compositions described and/or claimed in this document.

Computing device 500 includes a processor 502, memory 504, a storage device 506, a high-speed interface 508 connecting to memory 504 and high-speed expansion ports 510, and a low speed interface 512 connecting to low speed bus 514 and storage device 506. Each of the components 502, 504, 506, 508, 510, and 512, are interconnected using various busses, and can be mounted on a common motherboard or in other manners as appropriate. The processor 502 can process instructions for execution within the computing device 500, including instructions stored in the memory 504 or on the storage device 506 to display graphical information for a GUI on an external input/output device, such as display 516 coupled to high speed interface 508. In other implementations, multiple processors and/or multiple buses can be used, as appropriate, along with multiple memories and types of memory. Also, multiple computing devices 500 can be connected, with each device providing portions of the necessary operations, e.g., as a server bank, a group of blade servers, or a multi-processor system.

The memory 504 stores information within the computing device 500. In one implementation, the memory 504 is a volatile memory unit or units. In another implementation, the memory 504 is a non-volatile memory unit or units. The memory 504 can also be another form of computer-readable medium, such as a magnetic or optical disk.

The storage device 506 is capable of providing mass storage for the computing device 500. In one implementation, the storage device 506 can be or contain a computer-readable medium, such as a floppy disk device, a hard disk device, an optical disk device, or a tape device, a flash memory or other similar solid-state memory device, or an array of devices, including devices in a storage area network or other configurations. A computer program product can be tangibly embodied in an information carrier. The computer program product can also contain instructions that, when executed, perform one or more methods, such as those described above. The information carrier is a computer- or machine-readable medium, such as the memory 504, the storage device 506, or memory on processor 502.

The high-speed controller 508 manages bandwidth-intensive operations for the computing device 500, while the low speed controller 512 manages lower bandwidth intensive operations. Such allocation of functions is only an example. In one implementation, the high-speed controller 508 is coupled to memory 504, display 516, e.g., through a graphics processor or accelerator, and to high-speed expansion ports 510, which can accept various expansion cards (not shown). In the implementation, low-speed controller 512 is coupled to storage device 506 and low-speed expansion port 514. The low-speed expansion port, which can include various communication ports, e.g., USB, Bluetooth, Ethernet, wireless Ethernet can be coupled to one or more input/output devices, such as a keyboard, a pointing device, microphone/speaker pair, a scanner, or a networking device such as a switch or router, e.g., through a network adapter. The computing device 500 can be implemented in a number of different forms, as shown in the figure. For example, it can be implemented as a standard server 520, or multiple times in a group of such servers. It can also be implemented as part of a rack server system 524. In addition, it can be implemented in a personal computer such as a laptop computer 522. Alternatively, components from computing device 500 can be combined with other components in a mobile device (not shown), such as device 550. Each of such devices can contain one or more of computing device 500, 550, and an entire system can be made up of multiple computing devices 500, 550 communicating with each other.

The computing device 500 can be implemented in a number of different forms, as shown in the figure. For example, it can be implemented as a standard server 520, or multiple times in a group of such servers. It can also be implemented as part of a rack server system 524. In addition, it can be implemented in a personal computer such as a laptop computer 522. Alternatively, components from computing device 500 can be combined with other components in a mobile device (not shown), such as device 550. Each of such devices can contain one or more of computing device 500, 550, and an entire system can be made up of multiple computing devices 500, 550 communicating with each other.

Computing device 550 includes a processor 552, memory 564, and an input/output device such as a display 554, a communication interface 566, and a transceiver 568, among other components. The device 550 can also be provided with a storage device, such as a micro-drive or other device, to provide additional storage. Each of the components 550, 552, 564, 554, 566, and 568, are interconnected using various buses, and several of the components can be mounted on a common motherboard or in other manners as appropriate.

The processor 552 can execute instructions within the computing device 550, including instructions stored in the memory 564. The processor can be implemented as a chipset of chips that include separate and multiple analog and digital processors. Additionally, the processor can be implemented using any of a number of architectures. For example, the processor 510 can be a CISC (Complex Instruction Set Computers) processor, a RISC (Reduced Instruction Set Computer) processor, or a MISC (Minimal Instruction Set Computer) processor. The processor can provide, for example, for coordination of the other components of the device 550, such as control of user interfaces, applications run by device 550, and wireless communication by device 550. Processor 552 can communicate with a user through control interface 558 and display interface 556 coupled to a display 554. The display 554 can be, for example, a TFT (Thin-Film-Transistor Liquid Crystal Display) display or an OLED (Organic Light Emitting Diode) display, or other appropriate display technology. The display interface 556 can comprise appropriate circuitry for driving the display 554 to present graphical and other information to a user. The control interface 558 can receive commands from a user and convert them for submission to the processor 552. In addition, an external interface 562 can be provided in communication with processor 552, so as to enable near area communication of device 550 with other devices. External interface 562 can provide, for example, for wired communication in some implementations, or for wireless communication in other implementations, and multiple interfaces can also be used. The memory 564 stores information within the computing device 550. The memory 564 can be implemented as one or more of a computer-readable medium or media, a volatile memory unit or units, or a non-volatile memory unit or units. Expansion memory 574 can also be provided and connected to device 550 through expansion interface 572, which can include, for example, a SIMM (Single In Line Memory Module) card interface. Such expansion memory 574 can provide extra storage space for device 550, or can also store applications or other information for device 550. Specifically, expansion memory 574 can include instructions to carry out or supplement the processes described above, and can also include secure information. Thus, for example, expansion memory 574 can be provided as a security module for device 550, and can be programmed with instructions that permit secure use of device 550. In addition, secure applications can be provided via the SIMM cards, along with additional information, such as placing identifying information on the SIMM card in a non-hackable manner.

The memory can include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 564, expansion memory 574, or memory on processor 552 that can be received, for example, over transceiver 568 or external interface 562.

Device 550 can communicate wirelessly through communication interface 566, which can include digital signal processing circuitry where necessary. Communication interface 566 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through radio-frequency transceiver 568. In addition, short-range communication can occur, such as using a Bluetooth, Wi-Fi, or other such transceiver (not shown). In addition, GPS (Global Positioning System) receiver module 570 can provide additional navigation- and location-related wireless data to device 550, which can be used as appropriate by applications running on device 550.

Device 550 can also communicate audibly using audio codec 560, which can receive spoken information from a user and convert it to usable digital information. Audio codec 560 can likewise generate audible sound for a user, such as through a speaker, e.g., in a handset of device 550. Such sound can include sound from voice telephone calls, can include recorded sound, e.g., voice messages, music files, etc. and can also include sound generated by applications operating on device 550.

The computing device 550 can be implemented in a number of different forms, as shown in the figure. For example, it can be implemented as a cellular telephone 580. It can also be implemented as part of a smartphone 582, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations of such implementations. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. To provide for interaction with a user, the systems and techniques described here can be implemented on a computer having a display device, e.g., a CRT (cathode ray tube) or LCD (liquid crystal display) monitor for displaying information to the user and a keyboard and a pointing device, e.g., a mouse or a trackball by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the systems and techniques described here, or any combination of such back end, middleware, or front end components. The components of the system can be interconnected by any form or medium of digital data communication, e.g., a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other. A number of embodiments have been described. Nevertheless, it will be understood that various modifications can be made without departing from the spirit and scope of the invention. In addition, the logic flows depicted in the figures do not require the particular order shown, or sequential order, to achieve desirable results. In addition, other steps can be provided, or steps can be eliminated, from the described flows, and other components can be added to, or removed from, the described systems. Accordingly, other embodiments are within the scope of the following claims.

Embodiments of the disclosure and all of the functional operations described in this specification can be implemented in digital electronic circuitry, or in computer software, firmware, or hardware, including the structures disclosed in this specification and their structural equivalents, or in combinations of one or more of them. Embodiments of the methods and compositions can be implemented as one or more computer program products, e.g., one or more modules of computer program instructions encoded on a computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as a program, software, software application, script, or code) can be written in any form of programming language, including compiled or interpreted languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a tablet computer, a mobile telephone, a personal digital assistant (PDA), a mobile audio player, a Global Positioning System (GPS) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the disclosure can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the disclosure can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the methods, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other. While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

In each instance where an HTML file is mentioned, other file types or formats may be substituted. For instance, an HTML file may be replaced by an XML, JSON, plain text, or other types of files. Moreover, where a table or hash table is mentioned, other data structures (such as spreadsheets, relational databases, or structured files) may be used.

### EXAMPLES

The invention is further described in the following examples, which do not limit the scope of the invention described in the claims.

### Example 1: Benchmarking the Indel Quantification Method Using a Well-Characterized Nuclease

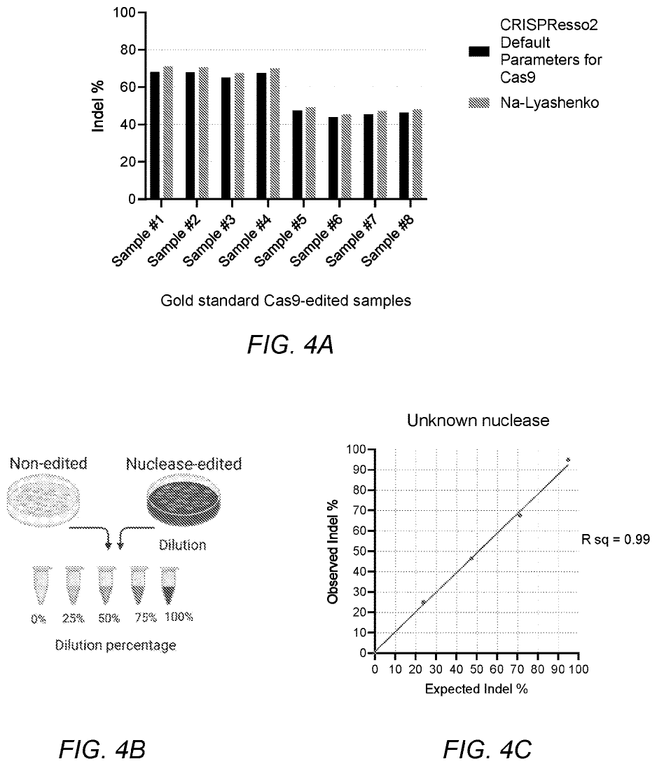

In order to evaluate the efficacy of the indel quantification methods disclosed herein, the data processing pipeline is applied to data generated by next-generation sequencing of mammalian cells edited by SpCas9, a Type II nuclease with a known cleavage site and editing pattern. The percentage of cells estimated to comprise an indel at the target DNA sequence after editing is determined by standard CRISPResso2 processing parameters, and by the methods disclosed herein. As shown in FIG. 4A, the methods disclosed herein perform similarly to the standard CRISPResso2 processing parameters, indicating that the method does not over- or underestimate the percentage of cells estimated to comprise an indel at the target DNA sequence after editing with a well-characterized nuclease with a known cleavage site and editing pattern.

### Example 2: Dilution Experiment to Quantify Indels

In order to validate the accuracy of the indel quantification methods disclosed herein, the data processing pipeline was applied to data generated by next-generation sequencing of mammalian cells edited by a Type V nuclease with an unknown cleavage site in a serial dilution experiment. DNA isolated from mammalian cells edited by the Type V nuclease was serially diluted with DNA isolated from non-edited cells in proportions of, for example, 0% non-edited/100% edited; 25% non-edited/75% edited; 50% non-edited/50% edited; 75% non-edited/25% edited; 0% non-edited/100% edited (FIG. 4B). The serially diluted DNA mixtures were sequenced to produce sequencing reads comprising the edited target sequence. As shown in FIG. 4C, observed indel percentages (y-axis) were plotted against expected indel percentages (x-axis) for each of the serial dilution concentrations. Linearity of the plot, with an R-squared value of approximately 0.99, confirmed the efficacy of the indel quantification data processing pipeline.

### Example 3: Quantifying Indels after Editing with Novel Nucleases

In order to validate the efficacy of the indel quantification methods disclosed herein for estimating indels in target sequences edited by novel nucleases and varying guide sequences, the data processing pipeline is applied to data generated by next-generation sequencing of mammalian cells edited a novel Type V nuclease, at escalating concentrations and with two different guides: guide sequence A1 and F2. As shown in FIG. 6, the data processing pipeline accurately estimates indel percentages of editing by the novel Type V nuclease at nuclease concentrations of 5 μM, 11 μM, and 22 μM for guide sequence A1 and F2. Additionally, the data processing pipeline is applied to data generated by next-generation sequencing of mammalian cells edited by nuclease AsCas12a to validate the efficacy of the indel quantification methods disclosed herein.

### Example 4: Dilution Experiment to Quantify Larger Insertions

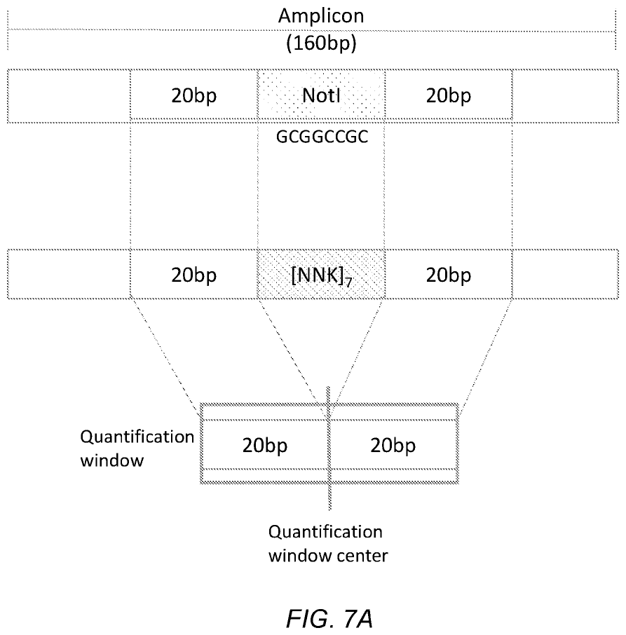

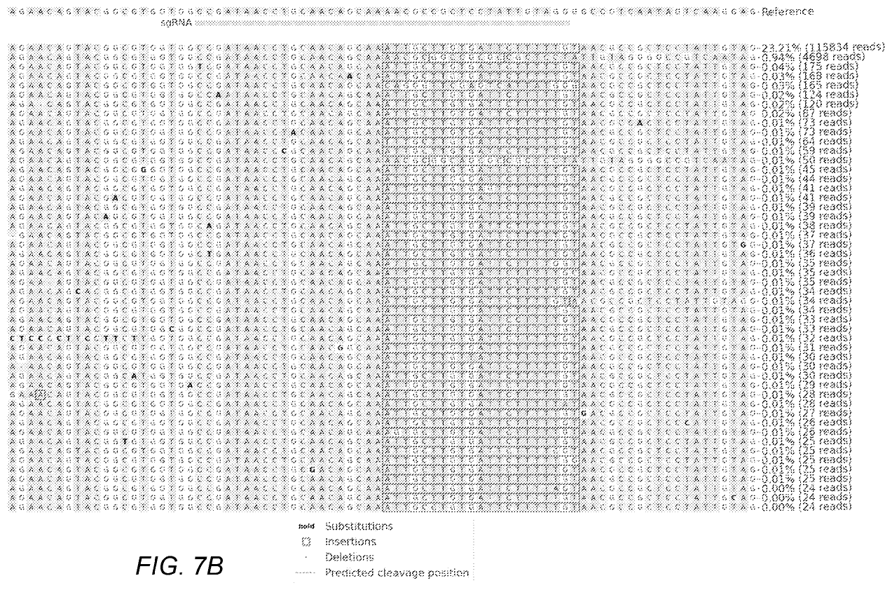

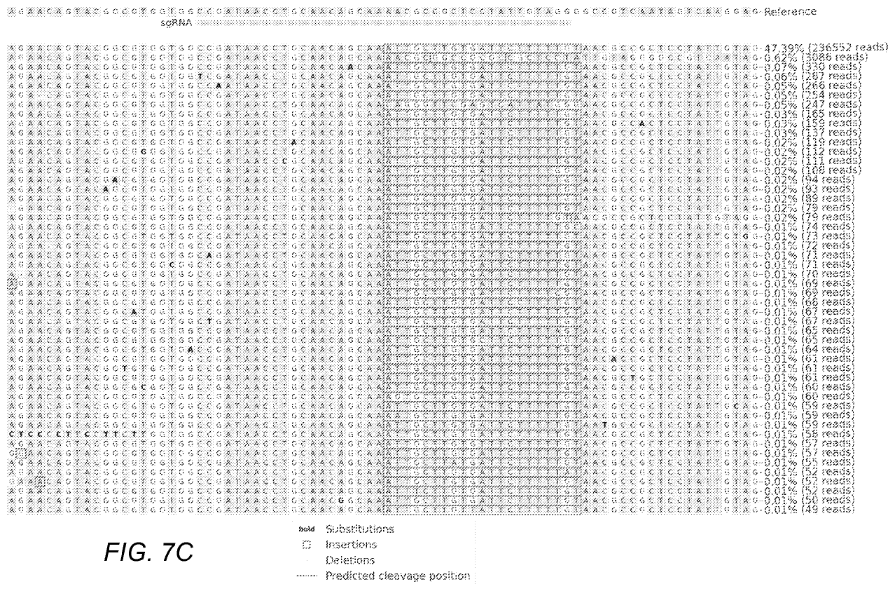

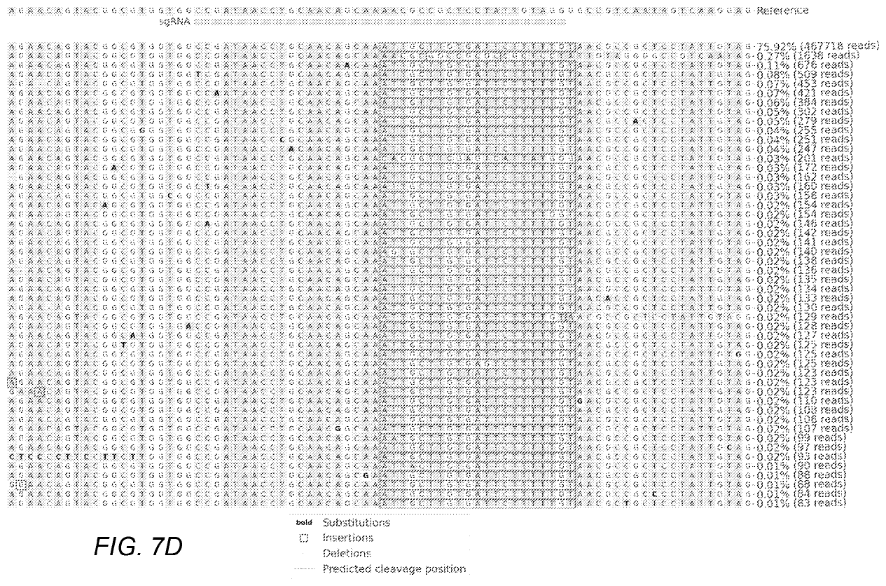

In order to validate the accuracy of the indel quantification methods disclosed herein for quantifying larger insertions, i.e., insertions larger than about 10 nucleotides, the data processing pipeline was applied to data generated by next-generation sequencing of mammalian cells where a sequence of known length, 21 nucleotides, was introduced at a particular insertion site in a serial dilution spike-in experiment (see, FIG. 7A). DNA isolated from mammalian cells comprising the insertion was serially diluted with DNA isolated from cells not comprising the insertion in proportions of, for example, 0% insertion/no insertion; 25% insertion/75% no insertion; 50% insertion/50% no insertion; 75% insertion/25% no insertion; 0% insertion/100% no insertion (FIG. 4B). The serially diluted DNA mixtures were sequenced to produce sequencing reads comprising the insertion site. As shown in FIG. 7B, for the experiment where the DNA comprising the insertion was spiked in at 25%, the spiked in sequence was detected at approximately 23%. As shown in FIG. 7C, for the experiment where the DNA comprising the insertion was spiked in at 50%, the spiked in sequence was detected at approximately 47%. As shown in FIG. 7D, for the experiment where the DNA comprising the insertion was spiked in at 75%, the spiked in sequence was detected at approximately 76%. These results indicate that the data processing pipeline disclosed herein is effective for quantifying insertions introduced by, e.g., CRISPR-mediated donor insertions, insertions mediated by homology directed repair (HDR), CRISPR-associated transposases (CAST) and other genome engineering methods as well as insertions for genomic libraries used in high throughput screening (HTS).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for quantifying insertions and/or deletions in a polynucleotide sequence caused by cleavage of a target polynucleotide sequence by a nucleic acid-guided nuclease, the method comprising:
in a computer system, receiving sample sequence data comprising a plurality of sequencing reads;
filtering the plurality of sequencing reads;
aligning the plurality of sequencing reads to a reference sequence;
defining a window based on a sequence location within a nucleic acid guide sequence and the locations of the ends of the nucleic acid guide sequence;
determining, based on the alignment of each sequencing read of the plurality of sequencing reads to the reference sequence, the number of sequencing reads comprising an insertion or deletion within the window relative to the reference sequence;
estimating, based on the number of sequencing reads comprising an insertion or deletion, the quantity of insertions and/or deletions in the polynucleotide sequence mediated by a nucleotide-directed nuclease.

2. The method of claim 1, wherein the nucleic acid-guided nuclease is a Class 2 nuclease.

3. The method of any one of claims 1-2, wherein the nucleic acid-guided nuclease is a type II nuclease.

4. The method of claim 3, wherein the nucleic acid-guided nuclease is SpCas9.

5. The method of claim 3, wherein the nucleic acid-guided nuclease is AsCas12a.

6. The method of any one of claims 1-2, wherein the nucleic acid-guided nuclease is a type V nuclease.

7. The method of any one of claims 1-6, wherein the nucleic acid guide is a guide RNA (gRNA).

8. The method of any one of claims 1-6, wherein the nucleic acid guide is a single guide RNA (sgRNA).

9. The method of any one of claims 1-8, wherein the center of the window is located at the center of the nucleic acid guide sequence.

10. The method of any one of claims 1-8, wherein the center of the window is located at the site where the nuclease cleaved the polynucleotide sequence.

11. The method of any one of claims 1-10, wherein the length of the window is equivalent to the length of the nucleic acid guide sequence.

12. The method of any one of claims 1-11, wherein the 5′ end of the window extends 50 basepairs 5′ to the 5′ end of the nucleic acid guide and the 3′ end of the window extends 50 basepairs 3′ to the 3′ end of the nucleic acid guide.

13. The method of any one of claims 1-12, wherein the 5′ end of the window extends 40 basepairs 5′ to the 5′ end of the nucleic acid guide and the 3′ end of the window extends 40 basepairs 3′ to the 3′ end of the nucleic acid guide.

14. The method of any one of claims 1-13, wherein the 5′ end of the window extends 30 basepairs 5′ to the 5′ end of the nucleic acid guide and the 3′ end of the window extends 30 basepairs 3′ to the 3′ end of the nucleic acid guide.

15. The method of any one of claims 1-14, wherein the 5′ end of the window extends 20 basepairs 5′ to the 5′ end of the nucleic acid guide and the 3′ end of the window extends 20 basepairs 3′ to the 3′ end of the nucleic acid guide.

16. The method of any one of claims 1-15, wherein the 5′ end of the window extends 10 basepairs 5′ to the 5′ end of the nucleic acid guide and the 3′ end of the window extends 10 basepairs 3′ to the 3′ end of the nucleic acid guide.

17. The method of any one of claims 1-16, wherein the 5′ end of the window extends 5 basepairs 5′ to the 5′ end of the nucleic acid guide and the 3′ end of the window extends 5 basepairs 3′ to the 3′ end of the nucleic acid guide.

18. The method of any one of claims 1-17, wherein the method further includes trimming of adapter sequences from the plurality of sequencing reads.

19. The method of any one of claims 1-18, wherein the plurality of sequencing reads are generated from targeted amplicon sequencing.

20. The method of any one of claims 1-19, wherein the plurality of sequencing reads are generated from targeted amplicon sequencing of DNA isolated from cells edited by the nucleic-acid guided nuclease.

21. The method of any one of claims 1-20, wherein the plurality of sequencing reads are paired-end sequencing reads.

22. The method of claim 21, wherein the method further comprises read merging of the paired-end sequencing reads to produce a single read for alignment to the reference sequence.

23. The method of claim 22, wherein the read merging of the paired-end reads further comprises applying a minimum paired-end read overlap score to the plurality of sequencing reads.

24. The method of claim 23, wherein the minimum paired-end read overlap score is 0, 10, 20, 30, 40, 50, 60, 70, 80, 90, or 100.

25. The method of any one of claims 23-24 wherein the minimum paired-end read overlap score is 10.

26. The method of any one of claims 22-25, wherein the read merging of paired-end reads further comprises applying a maximum paired-end read overlap score to the plurality of sequencing reads.

27. The method of claim 26, wherein the maximum paired-end read overlap score is 100, 110, 120, 130, 140, 150, 160, 170, 180, 190, 200, 210, 220, 230, 240, 250, 260, 270, 280, 290, or 300.

28. The method of any one of claims 26-27 wherein the maximum paired-end read overlap score is 100.

29. The method of any one of claims 1-28 wherein the filtering step further comprises applying a minimum average read quality score to the plurality of sequencing reads.

30. The method of claim 29, wherein the minimum average read quality score is 0, 5, 10, 15, 20, 25, 30, 35, or 40.

31. The method of any one of claims 1-30 wherein the filtering step further comprises applying a minimum single basepair score to the plurality of sequencing reads.

32. The method of claim 31, wherein the minimum single basepair score is 0, 1, 2, 3, 4, 5, 6, 7, 8, 9, 10, 11, 12, 13, 14, 15, 16, 17, 18, 19, or 20.

33. The method of any one of claims 1-32 wherein the aligning step further comprises applying an amplicon minimum alignment score to the plurality of sequencing reads.

34. The method of claim 33, wherein the amplicon minimum alignment score is 0, 10, 20, 30, 40, 50, 60, 70, 80, 90, or 100.

35. The method of any one of claims 1-34, wherein the nucleic acid-guided nuclease is a FokI nuclease.

36. The method of claim 35, wherein the FokI nuclease is fused to a transcription activator-like (TAL) protein.

37. The method of any one of claims 1-36, wherein the nucleic acid-guided nuclease is a zinc-finger nuclease.

38. A computer program product tangibly embodied on a computer-readable medium, comprising instructions that when executed by one or more processors are configured to:
receive sample sequence data comprising a plurality of sequencing reads;
filter the plurality of sequencing reads;
align the plurality of sequencing reads to a reference sequence;
define a window based on a sequence location within a nucleic acid guide sequence and the locations of the ends of the nucleic acid guide sequence;
determine, based on the alignment of each sequencing read of the plurality of sequencing reads to the reference sequence, the number of sequencing reads comprising an insertion or deletion within the window relative to the reference sequence; and
estimate, based on the number of sequencing reads comprising an insertion or deletion, the quantity of insertions and/or deletions in the polynucleotide sequence mediated by a nucleotide-directed nuclease.
